(19) United States
(12) Patent Application Publication
(10) Pub. No.: US 2025/0269013 A1
(43) Pub. Date: Aug. 28, 2025

(54) IMMUNOGENIC COMPOSITIONS FOR TREATMENT OF HEPATITIS B

(71) Applicant: Brii Biosciences Limited, Grand Cayman (KY)

(72) Inventors: David Evander Anderson, Newton, MA (US); Tanvir Ahmed, Ottawa (CA)

(73) Assignee: Brii Biosciences Limited, Grand Cayman (KY)

(21) Appl. No.: 18/883,612

(22) Filed: Sep. 12, 2024

Related U.S. Application Data
(63) Continuation of application No. 17/293,431, filed on May 12, 2021, now Pat. No. 12,128,100.
(60) Provisional application No. 62/760,439, filed on Nov. 13, 2018.

(51) Int. Cl. A61K 39/29; A61K 39/00; A61K 45/06; A61P 31/20; A61P 37/04

(52) U.S. Cl. CPC A61K 39/292; A61K 45/06; A61P 31/20; A61P 37/04; A61K 2039/545; A61K 2039/55505; A61K 2039/57

(57) ABSTRACT

The present disclosure provides compositions and methods useful for inducing a Th1 cell response in a subject suffering from Hepatitis B. As described herein, the compositions of the disclosure comprise HBsAg having S, Pre-S1 and Pre-S2 proteins and an aluminum phosphate adjuvant. In a preferred embodiment, the immunogenic composition comprises at least 20 μg/ml of HBsAg antigen and the amount of non-adsorbed antigen is at least 30%.

### CROSS REFERENCE TO RELATED APPLICATIONS

The present application is a continuation of U.S. application Ser. No. 17/293,431 filed May 12, 2021, which is the National Stage of International Application Number PCT/IB2019/001230 filed Nov. 13, 2019, which claims the benefit of U.S. Provisional Application No. 62/760,439, filed on Nov. 13, 2018, the contents of all of which are herein incorporated by reference in their entirety.

### REFERENCE TO SEQUENCE LISTING SUBMITTED VIA EFS-WEB

This application contains an electronic Sequence Listing which has been submitted in XML file format via Patent Center, the entire content of which is incorporated by reference herein in its entirety:

| File Name | Date of Creation | Size |
|---|---|---|
| 14874-006-999_SEQLISTING.xml | May 15, 2025 | 5,222 bytes |

### FIELD OF THE INVENTION

This invention is in the field of immunogenic compositions, in particular immunogenic compositions useful for treatment of Hepatitis B infection.

### BACKGROUND

Hepatitis B is a viral infection which is the causative agent of acute and chronic liver disease. It is transmitted in humans through contact with blood or other body fluids from an infected individual. Hepatitis B infection is a widespread and significant health problem causing chronic liver disease in over 250 million people and close to a million deaths a year worldwide (Global Hepatitis Report 2017, World Health Organization). Upon infection, Hepatitis B causes an acute phase of disease, which has no symptoms in most people. Those who do show symptoms experience jaundice, fatigue and abdominal pain with a small subset of sufferers experiencing acute liver failure which can be fatal. Following infection with Hepatitis B, the patient's immune system may either clear the virus resulting in a cure, or the patient may develop a chronic Hepatitis B infection. The consequences of chronic Hepatitis B are significant, since 30% of chronically infected adults eventually develop cirrhosis and/or liver cancer. The likelihood of developing a chronic disease decreases with age, with infants having a 90% chance of chronic infection, while healthy adults only have a 5%-10% chance.

Although vaccines for Hepatitis B infection have been pursued, a need exists for improved treatments for chronic Hepatitis B patients, particularly treatments that are able to elicit an enhanced cellular immune response in subjects.

### SUMMARY

Provided are various embodiments related to immunogenic compositions comprising an HBsAg antigen comprising S, Pre-S1 and Pre-S2 domains and an aluminum phosphate adjuvant, uses of the immunogenic compositions of the disclosure, methods of inducing a Th1 cell response by administering the immunogenic compositions of the disclosure and methods of treating Hepatitis B infection by administering the immunogenic compositions of the disclosure.

In some embodiments, the present disclosure provides an immunogenic composition comprising an HBsAg envelope antigen comprising S, Pre-S1 and Pre-S2 domains (e.g., S, Pre-S1 and Pre-S2 proteins described herein) and an aluminum phosphate adjuvant. In a preferred embodiment, the present disclosure provides an immunogenic composition comprising at least about 20 μg/ml (e.g., at least about 30 μg/ml, at least about 40 μg/ml, at least about 50 μg/ml, or at least about 60 μg/ml) of HBsAg envelope antigen comprising S, Pre-S1 and Pre-S2 domains and an aluminum phosphate adjuvant wherein the amount of unbound antigen is at least 30% (e.g., at least about 35%, at least about 40%, at least about 45%, at least about 50%, at least about 60%, at least about 70%, at least about 80%, or more). In a particularly preferred embodiment, the present disclosure provides a vaccine formulation comprising about 20 μg/ml-60 μg/ml (e.g., about 20 μg/ml, about 30 μg/ml, about 40 μg/ml, about 50 μg/ml, or about 60 μg/ml) of HBsAg antigen comprising S, Pre-S1 and Pre-S2 domains and an aluminum phosphate adjuvant wherein the amount of unbound antigen is at least about 30% (e.g., at least about 35%, at least about 40%, at least about 45%, at least about 50%, at least about 60%, at least about 70%, at least about 80%, or more).

In some embodiments, the immunogenic compositions of the disclosure comprise between 62.5 μg/ml and 500 μg/ml aluminum which is present as aluminum phosphate adjuvant. In a preferred embodiment, the immunogenic compositions of the disclosure comprise 500 μg/ml of aluminum as aluminum phosphate adjuvant. In a particularly preferred embodiment, the immunogenic composition of the disclosure comprises 500 μg/ml of aluminum as aluminum phosphate adjuvant and 20 μg/ml of HBsAg antigen having all three of the S, Pre-S1 and Pre-S2 proteins.

In another embodiment, the present disclosure provides an immunogenic composition for eliciting a Th1 response in a subject, said composition comprising HBsAg envelope antigens comprising S, Pre-S1 and Pre-S2 proteins and an aluminum phosphate adjuvant. In a preferred embodiment, the present disclosure provides an immunogenic composition for eliciting a Th1 response in a mammal, said composition comprising at least 20 μg/ml of HBsAg envelope antigens comprising S, Pre-S1 and Pre-S2 proteins and an aluminum phosphate adjuvant wherein the amount of unbound antigen is at least 30%. In a particularly preferred embodiment, the present disclosure provides an immunogenic composition for eliciting a Th1 response in a mammal, said composition comprising 20 μg/ml-60 μg/ml of HBsAg antigen comprising S, Pre-S1 and Pre-S2 proteins and an aluminum phosphate adjuvant wherein the amount of unbound antigen is at least 30%. In some embodiments, the immunogenic composition for eliciting a Th1 response in a mammal comprises between 62.5 μg/ml and 500 μg/ml aluminum which is present as aluminum phosphate adjuvant. In a preferred embodiment, the immunogenic composition for eliciting a Th1 response in a mammal comprises 500 μg/ml of aluminum as aluminum phosphate adjuvant and 20 μg/ml-60 μg/ml of HBsAg antigen comprising S, Pre-S1 and Pre-S2 proteins. In a particularly preferred embodiment, the immunogenic composition for eliciting a Th1 response in a mammal comprises 500 μg/ml of aluminum as aluminum phosphate adjuvant and 20 μg/ml of HBsAg antigen comprising S, Pre-S1 and Pre-S2 proteins.

In another embodiment, the present disclosure provides an immunogenic composition for treatment of Hepatitis B in a human subject in need thereof, said composition comprising HBsAg envelope antigens comprising S, Pre-S1 and Pre-S2 proteins and an aluminum phosphate adjuvant. In a preferred embodiment, the present disclosure provides an immunogenic composition for treatment of Hepatitis B in a human subject in need thereof, said composition comprising at least 20 μg/ml of HBsAg envelope antigen comprising S, Pre-S1 and Pre-S2 proteins and an aluminum phosphate adjuvant wherein the amount of unbound antigen is at least 30%. In a particularly preferred embodiment, the present disclosure provides an immunogenic composition for treatment of Hepatitis B in a human subject in need thereof, said composition comprising 20 μg/ml-60 μg/ml of HBsAg antigens comprising S, Pre-S1 and Pre-S2 proteins and an aluminum phosphate adjuvant wherein the amount of unbound antigen is at least 30%. In some embodiments, the immunogenic composition for treatment of Hepatitis B in a human subject in need thereof comprises 62.5 μg/ml to 500 μg/ml aluminum which is present as aluminum phosphate adjuvant. In a preferred embodiment, the immunogenic compositions for treatment of Hepatitis B in a human subject in need thereof comprises 500 μg/ml of aluminum as aluminum phosphate adjuvant and 20-60 μg/ml μg/ml of HBsAg antigens comprising S, Pre-S1 and Pre-S2 proteins. In a particularly preferred embodiment, the immunogenic compositions for treatment of Hepatitis B in a human subject in need thereof comprises 500 μg/ml of aluminum as aluminum phosphate adjuvant and 20 μg/ml μg/ml of HBsAg antigens comprising S, Pre-S1 and Pre-S2 proteins.

The present disclosure also encompasses the use of at least one of the immunogenic compositions of the disclosure in the preparation of a pharmaceutical composition intended for treating Hepatitis B infections.

The present disclosure further provides pharmaceutical compositions comprising the immunological compositions of the disclosure for administration to a subject in need thereof.

The present disclosure also provides a method for inducing a Th1 response in a mammal comprising administering a therapeutically effective amount of a composition of the disclosure.

The present disclosure also provides a method for the treatment of Hepatitis B infections, in particular chronic Hepatitis B infection, comprising administering to a subject in need thereof of a therapeutically effective amount of a composition of the disclosure.

In any of the aspects or embodiments described herein, a composition can comprise an S protein (e.g., an S protein comprising or consisting of an amino acid sequence having about 80%, 85%, 90%, 91%, 92%, 93%, 94%, 95%, 96%, 97%, 98%, 99%, or 100% identity to the amino acid sequence of SEQ ID NO:1); a Pre-S2 protein (e.g., a Pre-S2 protein comprising or consisting of an amino acid sequence having at least 80%, 85%, 90%, 91%, 92%, 93%, 94%, 95%, 96%, 97%, 98%, 99%, or 100% identity to the amino acid sequence of SEQ ID NO: 2); and a Pre-S1 protein (e.g., a Pre-S1 protein comprising or consisting of an amino acid sequence having at least 80%, 85%, 90%, 91%, 92%, 93%, 94%, 95%, 96%, 97%, 98%, 99%, or 100% identity to the amino acid sequence of SEQ ID NO:3). Embodiments described herein may comprise about 75-90% by weight S protein, about 2-8% by weight Pre-S1 protein, and about 5-15% by weight of Pre-S2 protein (e.g., 83+/−3.3% S protein, 6+/−3% Pre-S1 protein, and 11+/−3% Pre-S2 protein).

Other features, objects, and advantages of the present disclosure are apparent in the detailed description that follows. It should be understood, however, that the detailed description, while indicating embodiments of the present disclosure, is given by way of illustration only, not limitation. Various changes and modifications within the scope of the disclosure will become apparent to those skilled in the art from the detailed description.

### Listing of Sequences

The following is a list of sequences referred to herein:

SEQ ID NO: 1 is a Small Protein of HBSAG Amino
Acid Sequence
1 11 21 31
MENITSGFLG PLLVLQAGFF LLTRILTIPQ SLDSWWTSLN
41 51 61 71
FLGGSPVCLG QNSQSPTSNH SPTSCPPICP GYRWMCLRRF
81 91 101 111
IIFLFILLLC LIFLLVLLDY QGMLPVCPLI PGSTTTSTGP
121 131 141 151
CKTCTTPAQG NSMFPSCCCT KPTDGNCTCI PIPSSWAFAK
161 171 181 191
YLWEWGSVRF SWLSLLVPFV QWFVGLSPTV WLSVIWMMWY
201 211 221
WGPNLYNILS PFIPLLPIFF CLWVYI
SEQ ID NO: 2 is a Medium Protein of HBSAG Amino
Acid Sequence
1 11 21 31
MQWNSTAFHQ ALQHPRVRGL YFPAGGSSSG TVNPAQNIAS
41 51 61 71
HISSISSRTG DPAPNMENIT SGFLGPLLVL QAGFFLLTRI
81 91 101 111
LTIPQSLDSW WTSLNFLGGS PVCLGQNSQS PTSNHSPTSC
121 131 141 151
PPICPGYRWM CLRRFIIFLF ILLLCLIFLL VLLDYQGMLP
161 171 181 191
VCPLIPGSTT TSTGPCKTCT TPAQGNSMFP SCCCTKPTDG
201 211 221 231
NCTCIPIPSS WAFAKYLWEW GSVRFSWLSL LVPFVQWFVG
241 251 261 271
LSPTVWLSVI WMMWYWGPNL YNILSPFIPL LPIFFCLWVY
281
I
SEQ ID NO: 3 is a Large Protein of HBSAG Amino
Acid Sequence:
MGLSWTVPLE WGKNQSTSNP LGFFPDHQLD PAFGANSNNP
DWDLNSNKDH WPQANQVGVG AFGPGFTPPH GGLLGWSSQA
QGTLHTVPAV PPPASTNRQT KRQPTPISPP LRDSHPQAMQ
WNSTAFHQAL QHPRVRGLYF PAGGSSSGTV NPAQNIASHI
SSISSRTGDP APNMENITSG FLGPLLVLQA GFFLLTRILT
IPQSLDSWWT SLNFLGGSPV CLGQNSQSPT SNHSPTSCPP
ICPGYRWMCL RRFIIFLFIL LLCLIFLLVL LDYQGMLPVC
PLIPGSTTTS TGPCKTCTTP AQGNSMFPSC CCTKPTDGNC
TCIPIPSSWA FAKYLWEWGS VRFSWLSLLV PFVQWFVGLS
PTVWLSVIWM MWYWGPNLYN ILSPFIPLLP IFFCLWVYI

### Detailed Description of the Embodiments

The inventors of the present application have made an improved therapeutic Hepatitis B vaccine which is effective at inducing a Th1 cell immune response.

The Hepatitis B virus is a member of the genus Orthohepadnavirus, of the Hepadnaviridae family of viruses. It is a small, enveloped DNA virus comprising a nucleocapsid and an outer envelope of Hepatitis B surface antigen (HBsAg). The HBsAg antigen is composed of three related envelope proteins that are all encoded by the same open reading frame on the viral DNA (the “S ORF”). These three proteins have the same C terminus but differ at their N-termini due to the presence of three in-frame ATG start codons that divide the S ORF into three regions. The S or “small” envelope protein is the most abundant and the smallest with 226 amino acids. The M or “middle” surface protein includes the S protein and has an extra protein domain consisting of 55 amino acids known as pre-S2. The protein known as the L or “large” protein consists of the S, the Pre-S2 and a third protein domain is known as pre-S1 which has 118 amino acids. The viral genome of Hepatitis B is prone to replication errors, with the result that many genotypes and genetic variants exist. At least ten different genotypes of the virus have been identified, along with many mutations, some of which occur in the S and Pre-S domains. Therefore, many sequence variations exist for each of the three protein domains.

In some embodiments, an S protein described herein comprises or consists of the amino acid sequence of SEQ ID NO:1. In some embodiments, an S protein described herein comprises or consists of an amino acid sequence having at least 80%, 85%, 90%, 91%, 92%, 93%, 94%, 95%, 96%, 97%, 98%, or 99% identity to the amino acid sequence of SEQ ID NO:1. In some embodiments, a Pre-S2 protein described herein comprises or consists of the amino acid sequence of SEQ ID NO:2. In some embodiments, a Pre-S2 protein described herein comprises or consists of an amino acid sequence having at least 80%, 85%, 90%, 91%, 92%, 93%, 94%, 95%, 96%, 97%, 98%, or 99% identity to the amino acid sequence of SEQ ID NO:2. In some embodiments, a Pre-S1 protein described herein comprises or consists of the amino acid sequence of SEQ ID NO:3. In some embodiments, a Pre-S1 protein described herein comprises or consists of an amino acid sequence having at least 80%, 85%, 90%, 91%, 92%, 93%, 94%, 95%, 96%, 97%, 98%, or 99% identity to the amino acid sequence of SEQ ID NO:3.

No effective treatment for Hepatitis B infection has been found. Acute infection is treated with bedrest. Chronic infection is usually treated with antiviral agents, specifically nucleoside-based antiviral agents such as tenofovir, lamivudine, or entecavir or with pegylated interferon alpha (PEG-IFN a). The antiviral agents can slow the progression of cirrhosis and reduce the incidence of liver cancer, but they have proven to be unable to achieve clearance of the virus from the patient. Furthermore, they are costly and they must be continued for the life of the patient to maintain effectiveness. PEG-IFN a is associated with side effects and leads to a sustained antiviral response in only about 30% patients. Accordingly, medicinal treatments for chronic Hepatitis B are generally only effective to slow the progress of the disease, and have not been successful as cures. In order to clear the Hepatitis B virus, and thereby avoid a chronic condition, a patient must generate a potent and diverse immune response. In particular, a strong and specific T cell response is essential to achieving viral clearance of Hepatitis B (Bertoletti and Rivino (2014) Curr Opin Infect. Dis 27:528-534). This type of response is seen almost exclusively in adult patients with mature immune systems (Bertoletti and Gehring, (2013) PLOS Path. 9:1-4).

Given the significant health impact of Hepatitis B infection, much attention has focused on prevention of the disease. Prophylactic vaccines against Hepatitis B have been available for over 30 years and, in many countries, universal vaccination against Hepatitis B is conducted on children. The first prophylactic vaccines against Hepatitis B were developed in the late 1970's using plasma from Hepatitis B patients. Improved Hepatitis B vaccines became available in the 1980's and were produced in yeast cells using recombinant DNA technology to express the S-domain of HBsAg. These more modern vaccines quickly supplanted the first vaccines and are still the principal commercial vaccines on the market today. A more advanced Hepatitis B vaccine was developed in the 1990s, which is produced in mammalian cells and contains all three domains of HBsAg, specifically S, Pre-S1 and Pre-S2. The presence of the Pre-S1 and Pre-S2 domains has been associated with enhanced immunogenic response and, as well, inhibition of viral binding and infectivity (U.S. Pat. No. 5,242,812). The biologic function of the various envelope proteins and the nature of the immune response to each of the components (S, pre-S2, and pre-S1) are only partially understood. However, it is known that, in acute Hepatitis B infection, Pre-S1 and pre-S2 antigens and antibodies appear and disappear early after acute infection, and that antibodies to the S domain appear several weeks later. This, as well as studies using either the Pre-S1 or the Pre-S2 domain antigens, have suggested that the pre-S1 and pre-S2 antigens may be implicated in inducing T-cell help for production of antibodies against the S domain (Milich et al (1985) PNAS 82 8170-8172; Milich et al (1986) J Immunol 137 315-322).

Commercially available prophylactic Hepatitis B vaccines have proven to successfully induce a strong antibody response against the S protein domain of HBSAG in healthy subjects, particularly those who are young and have no impairment to their immune system. However, attempts to use these vaccines as therapeutic treatments in chronic Hepatitis B patients have proved unsuccessful. Clinical trials conducted to measure the therapeutic effect of Hepatitis B vaccines containing the HBSAG S domain alone and the Pre-S2 and S domains together showed that the vaccines were unable achieve clearance of the Hepatitis B virus (Pol et al, (2001) J. Hepatol 34:917-921). The failure of immunogenic treatment to treat chronic Hepatitis B infection is generally attributed to “immune tolerance”, a phenomenon whereby a Hepatitis B patient's immune system becomes unresponsive to further exposure to Hepatitis B antigens, particularly the HBsAg. Immune tolerance in chronic Hepatitis B patients has been associated with the virus' large production of Hepatitis B antigens, which causes exhaustion of T cells, specifically Hepatitis B specific CD8+ T cells (Boni, C. et al, (2007) J. Virol. 81:4215-4225). Exhausted Hepatitis B specific T cells hyper-express the programmed cell death protein 1 (PD-1) which promotes death of antigen-specific T cells. In patients with high loads of Hepatitis B virus, the destruction of antigen specific T cells can lead to the complete disappearance of these cells from the liver. The result is a weakened T cell response to stimulation by Hepatitis B antigens.

In addition to the challenge presented by immune tolerance, Hepatitis B vaccines containing only the HBsAg S protein may fail to effect clearance of the virus from chronic patients due to their weak ability to stimulate cellular immunity by a T cell response in most subjects. In an effort to more effectively stimulate a T cell response, therapeutic vaccination studies have been carried out in chronic Hepatitis B patients using Hepatitis B vaccines which contain all three of the Pre-S1, Pre-S2 and S protein domains. These studies showed a Hepatitis B specific T cell response in some patients. However, the effect was transient and did not lead to clearance of the disease, possibly because the vaccine stimulated a Th2 response but did not stimulate a CD8+ T-lymphocyte response. (Jung et al, (2002) Vaccine 20:3598-3612, Kosinka et al (2015) Med. Microbiol. Immunol 204). Th2 cells, characterized by secretion of IL-4, IL-5, and IL-13 cytokines, promote antibody production and are generally associated with allergic responses and are less effective against viral infections than Th1 cells, characterized by secretion of IFN-γ. Further attempts to induce a successful immunological response using DNA vaccines also failed to achieve a sustained response (Bertoletti and Gehring (2009) Exp. Rev, Gastro. Hep. 3:561-569).

Many researchers have concluded that a complete cure for Hepatitis B is many years away and that, by using a combination of therapies, it may be possible to achieve what has been termed a “functional cure”, whereby chronic patients achieve a sustained reduction in Hepatitis B virus and other markers of disease and a reduced incidence of liver cancer following cessation of treatment (Lok et al, (2017) J. Hep. 67:847-861). Due to the failure of antiviral treatments and prophylactic vaccines alone to achieve a functional cure, some researchers have concluded that it may be necessary to combine prophylactic vaccines with Hepatitis B treatments which reduce viral load. Reduction in viral load may reduce the level of immune tolerance and thereby improve the ability of the vaccine treatment to induce an immune response which is able to control the infection (Zoulim, F. et al (2018) Cold Spring Harb. Perspect. Med. (2015): 5: a21501). For example, combinations of prophylactic vaccines with nucleoside inhibitors have been attempted. However, to date, combination therapies using commercially available Hepatitis B vaccines have not succeeded in achieving viral clearance from chronic Hepatitis B patients (Vandepapeliere et al (2007) Vaccine 51:8585-8597). In a Vietnamese study, an attempt to improve responsiveness was made by combining a newer vaccine, Genhevac B (a Pre-S1, Pre-S2 and S protein vaccine with aluminum hydroxide adjuvant), with a nucleoside inhibitor (lamivudine). This combination was superior to the individual therapies alone in reducing levels of viral DNA in patients. However, the effect was not sustained after the vaccine treatments were discontinued. Accordingly, this combination treatment failed to recruit the patients' immune system to effectively clear the virus (Hoa, (2009) Antimicrob. Agents and Chemo. 53:5134-5140).

More complex triple combination treatments have been proposed that include an antibody that blocks PD-1 activity along with a nucleoside inhibitor and a prophylactic Hepatitis B vaccine. A study of this triple combination in a woodchuck model using a virus similar to Hepatitis B was effective in only a third of the animals (Kosinka et al, (2015) supra: 103-114; Liu et al, Virologica, (2014) 29:10-16). Furthermore, the efficacy of this triple combination has not been demonstrated in humans, and it entails the use of costly treatments, possibly indefinitely. Accordingly, the use of conventional prophylactic vaccines for therapeutic purposes, even in combination with other drugs, has not proven successful as a functional cure for Hepatitis B in chronic patients. Therefore, to achieve a sustained response in chronic Hepatitis B patients, improved means must be found to enhance the ability of these patients to mount a T cell response against the virus.

Newer strategies for treatment of chronic Hepatitis B have emerged which utilize gene silencing approaches. Examples of these treatments include the use of small interfering RNA (SiRNA) to interfere with viral gene expression, in particular HBsAg. Like nucleoside inhibitors, these therapies have the potential to reduce viral load and levels of secreted HBsAg (Woodell et al, (2015) Mol. Ther. 21:973-985) and it has been suggested that they may be useful in combination with treatments that induce an immune response (Ren et al. (2013) PLOSone 8 (3): e57525). However, the effectiveness of this combination has not been established and, given the failure of earlier studies with antiviral agents, the effectiveness may depend on the combination including an immunological agent which is capable of stimulating a robust Th1 cell response.

Previous attempts have been made to enhance the T cell response to Hepatitis B vaccines by altering vaccine formulations. Most commercially available Hepatitis B vaccines, including the widely used Hepatitis B vaccine, Engerix B®, and the Hepatitis B vaccine containing S/Pre-S1 and Pre-S2, Sci-B-Vac,® are formulated with aluminum hydroxide adjuvant. Aluminum hydroxide adjuvant is one of a family of aluminum containing adjuvants that are sometimes informally referred to as “alum” (although the word “alum” is more correctly used to describe hydrated potassium aluminum sulfate (KAl(SO₄)₂·12 H₂O) which was used as an early adjuvant for diphtheria toxoid but abandoned due to difficulties with manufacturing). Aluminum-based adjuvants have been used in human vaccines since 1932 and, although they have a long record of safety, their mode of action is not completely understood. It is generally believed that aluminum-based adjuvants enhance immune response by activation of dendritic cells.

Studies have been conducted to better understand and enhance the effectiveness of aluminum-based adjuvants. The two most frequently used aluminum-based adjuvants are referred to as “aluminum phosphate” and “aluminum hydroxide”, with aluminum hydroxide adjuvant being the most widely used commercially. Aluminum hydroxide adjuvant is not Al(OH)₃, but rather crystalline aluminum oxyhydroxide (AlOOH) which has a larger surface area than crystalline aluminum hydroxide. Aluminum phosphate adjuvant is actually amorphous aluminum hydroxy phosphate (Al(OH)ₓ(PO4)_{y}) in which some of the hydroxyl groups of aluminum hydroxide adjuvant are replaced by phosphate groups. The surface of aluminum phosphate adjuvant is composed of Al—OH and Al—OPO₃ groups. Although they are chemically similar, the two adjuvants have different chemical properties. Aluminum hydroxide adjuvant has a crystalline structure, a large surface area and a positive charge at neutral pH (+30 m Volts). Aluminum phosphate adjuvant is amorphous and has a negative charge at neutral pH (approx.−20 m Volts). Also, aluminum phosphate adjuvant has been shown to dissolve more readily following injection.

Antigens “adsorb” onto aluminum-based adjuvants, meaning that they adhere to the adjuvant forming a layer on the surface. Antigens adsorb onto the surface of aluminum-based adjuvants through electrostatic interaction, van der Waals forces and ligand (principally phosphate) exchange. Ligand exchange is the strongest adsorption force and can occur even when an electrostatic repulsive force is present. HBsAg is largely composed of phospholipids which contain phosphate groups that adsorb strongly to the hydroxylated mineral surface of the adjuvant by ligand exchange between the phosphate groups in the antigen and the surface hydroxyls in the aluminum-based adjuvant. Electrostatic attraction is not the predominant adsorption force for HBsAg (Iver et al, (2004) Vaccine, 22:1475-1479).

Traditionally, adsorption of antigen to aluminum-based adjuvants was intentionally maximized because adsorption was generally thought the be important for immunostimulatory effect due to retention of the antigen at the injection site, creating a “depot” effect with prolonged release of the antigens to the immune system. Moreover, complete adsorption of antigen leads to greater long-term stability of the vaccine formulation during long-term storage. Engerix B® contains 10 μg HBsAg per 250 μg aluminum from aluminum hydroxide adjuvant. This ratio, 0.04 μg HBsAg/μg aluminum, ensures complete adsorption. However, more recently, the relationship between antibody and T cell immunity, efficacy, and adsorption has become less clear. Studies on the effect of the tightness of binding of HBsAg to aluminum hydroxide on antibody production in immunized mice have indicated that the vaccine formulations with the tightest adsorption of the antigen onto the adjuvant yielded the lowest antibody response. (Clapp et al (2011) J. Pharm Sci 100 (2) 388-401). One study using the HBsAg S antigen domain showed that aluminum-based adjuvants having a higher phosphate content show decreased adsorption, weaker antigen binding and greater antibody response (Egan et al, (2009) Vaccine 27:3175-3180). Similarly, a single antigen Hepatitis B vaccine containing aluminum phosphate adjuvant elicited a greater antibody response than a similar vaccine formulated with aluminum hydroxide adjuvant and was effective at a lower adjuvant concentration (Fazeli et al, (2008) DARU 3:143-148). At least one study has shown that adsorption of antigen by an aluminum-based adjuvant is not required in order for the adjuvant to enhance the antibody response, leading to the conclusion that inflammation and not adsorption is related to immunogenicity (Noe et al, (2010) Vaccine 28:3588-3594). Little is known about the extent to which altered aluminum compositions and amounts impact Th2 vs. Th1 T cell immunity, though studies reporting higher antibody responses would be assumed by those skilled in the art to have promoted enhanced Th2 but not Th1 responses.

Although aluminum-based adjuvants are well known to improve immunogenicity, it is also well known that they act primarily to enhance antibody production and therefore are most effective at targeting pathogens killed or inhibited through interactions with antibodies. They are generally regarded as ineffective to elicit a Th1 response and rather induce an inflammatory, Th2, response by improving the attraction and uptake of antigen by antigen-presenting cells (APCs). Therefore, aluminum-based adjuvants have not been employed in the search for better stimulants of Th1 cell immunity.

In order to improve the cellular, or “innate” immune response, more modern adjuvants have been added to Hepatitis B vaccines. One such modern adjuvant is the endotoxin, monophosphoryl lipid A (MPL), a bacterial liposaccharide. WO 93/19780, discloses stimulation of T cell responses by HBsAg vaccines with a combination of adjuvants. No T cell response (as measured by IL-2, IFN-γ and IL-4) was observed in Balb/c mice following immunization with HBsAg absorbed on aluminum hydroxide. However, when 3-de-O-acylated monophosphoryl Lipid A (3D-MPL) was added to the formulation, a Th1 cell response was observed (as measured by IL-2 and IFN—Y). A subsequently filed patent by the same research group, U.S. Pat. No. 5,972,346, disclosed improved humoral immunogenicity when aluminum phosphate was used instead of aluminum hydroxide in an HBsAg/3D MPL vaccine formulation. This discovery lead to the development of a commercial vaccine sold under the brand name Fendrix®. Subsequent studies showed that Fendrix elicits a Th1 cellular immune response. However, Fendrix is a costly product, and it has only been approved for use in Europe in patients over 15 years of age suffering from renal deficiency. Furthermore, in other studies, the effects of the 3D-MPL adjuvant have been shown to be short-lived and mostly limited to the site of injection and regional lymph nodes (De Pasquale et al., Vaccine 2015).

More recently, US20160136264 disclosed a Hepatitis B vaccine consisting of hepatitis B antigen fragments and an adjuvant consisting of immunomodulatory DNA sequences that include CpG motifs which was able to mediate a Th1 response in mice. However, safety concerns have been raised about the use of CpG-based adjuvants in Hepatitis B vaccines and marketing authorization has not be obtained in all jurisdictions.

The inventors of the present application have found that an immunogenic composition comprising of all three HBsAg proteins (S, Pre-S1 and Pre S-2) and an aluminum phosphate adjuvant is effective at stimulating a Th1 cell response in a mammal when the composition has a significant amount of unbound antigen. Physicochemical analysis of the compositions of the disclosure shows that the adjuvant is weakly bound to the HBsAg antigens. As demonstrated further in the Examples herein, this binding is significantly weaker than seen in Hepatitis B vaccines formulated with aluminum hydroxide adjuvant. It is also significantly weaker than seen in formulations comprising HBsAg antigen and aluminum phosphate adjuvant having a lower ratio of HBsAg antigen to aluminum phosphate adjuvant.

Surprisingly, the compositions of the disclosure induced an enhanced Th1 cell response in a mammal without the presence of another, more modern, adjuvant which is known to stimulate cellular immunity such as MPL or CpG. These Th1 cell responses were demonstrated experimentally using different immunological markers, specifically antigen-specific IFN-γ responses and IgG2a/IgG1 ratios. This is surprising in view of previous studies which have shown no Th1 response to conventional Hepatitis B vaccines formulated with aluminum-based adjuvants alone.

Even more surprising, the immunogenic compositions of the disclosure are effective at enhancing Th1 cell responses when a significantly reduced concentration of aluminum phosphate adjuvant was used relative to the amount of antigen in the composition, as compared to a commercially available prophylactic vaccine. In particular, the compositions of the disclosure are effective at enhancing Th1 responses with a 50% lower ratio of aluminum phosphate adjuvant to antigen as compared to the most widely used commercially available prophylactic vaccine.

The vaccines of the disclosure comprise HBsAg which includes all three of the S, Pre-S1 and Pre-S2 proteins. The HBsAg may originate from any genotype, strain or isolate of Hepatitis B. Further, the HBsAg may originate from a native HBsAg or from a modified HBsAg. HBsAg antigen may be isolated from a natural source of Hepatitis B virus such as biological samples (e.g. blood, plasma, sera, semen, saliva, tissue sections, biopsy specimen etc.) collected from an infected subject, cultured cells or tissue cultures. HBsAg may also be produced using recombinant techniques in cells. In some embodiments, HBsAg is expressed in a mammalian cell line. In some embodiments, HBsAg is expressed in Chinese Hamster Ovary cell lines. HBsAg antigen comprising S, Pre-S1 and Pre-S2 domains may be produced using the method disclosed in U.S. Pat. No. 5,242,812. Nucleotide sequences encoding different HBsAg may be found in data banks such as Genbank and in the published literature (for example Fukimori et al (1990) 18 Nuc. Acid Res 4587; Vaudin et al (1988) 69 J. Gen Virol. 1383-1389). An amino acid sequence for the large protein of HBsAg is shown in SEQ ID NO. 1. In a preferred embodiment, the immunogenic compositions of the disclosure comprise 20 μg/ml or more of HBsAg antigen having all three of the S, Pre-S1 and Pre-S2 proteins. In another preferred embodiment, the immunogenic composition of the disclosure comprises 20 μg/ml-60 μg/ml of HBsAg antigen having all three of the S, Pre-S1 and Pre-S2 proteins.

The vaccine formulations of the present disclosure further comprise aluminum phosphate adjuvant. One example of an aluminum phosphate adjuvant suitable for use in the present disclosure is Adju-Phos®, an aluminum phosphate wet gel suspension manufactured by Brenntag.

As shown in Example 2, HBsAg vaccine formulations using aluminum hydroxide adjuvant (including the commercially available, prophylactic formulation of the vaccine containing all three of the S, Pre-S1 and Pre-S2 antigens, sold in association with the trademark Sci-B-Vac) contain less than 5% of non-adsorbed antigen. As shown in Table 3, a formulation containing the same amount of antigen used in the prophylactic vaccine, 10 μg/ml of HBsAg antigen having all three of the S, Pre-S1 and Pre-2 domains, with aluminum phosphate adjuvant rather than aluminum hydroxide did not have an increased amount of non-adsorbed antigen and, in fact, as shown in Table 3, there was no unabsorbed antigen in this formulation. This was surprising because aluminum phosphate adjuvant is known to bind more weakly to HBsAg antigen than aluminum hydroxide adjuvant. Nevertheless, at an antigen concentration of 10 μg/ml of HBsAg, similar levels of adsorption were seen using both the aluminum phosphate adjuvant and the aluminum hydroxide adjuvant.

However, surprisingly, a formulation having an increased concentration of HBsAg antigen (comprising S, Pre-S1 and Pre-S2 proteins) and the same concentration of aluminum phosphate adjuvant demonstrated a substantially increased content of non-adsorbed antigen. An aluminum content of 500 μg/ml from aluminum phosphate adjuvant is equivalent to 2.27 mg/ml of aluminum phosphate adjuvant. At an HBsAg concentration of 20 μg/ml and 500 μg/ml of aluminum present as aluminum phosphate adjuvant, the amount of non-adsorbed antigen was 54.8%. At an HBsAg concentration of 40 μg/ml and 500 μg/ml of aluminum as aluminum phosphate adjuvant, the amount of non-adsorbed antigen was 35.8%. At an HBsAg concentration of 60 μg/ml and 500 μg/ml of aluminum as aluminum phosphate adjuvant, the amount of non-adsorbed antigen was 47.4%. The lowered antigen adsorption was not observed in formulations containing the same HBsAg antigen concentration with aluminum hydroxide adjuvant. In fact, when aluminum hydroxide adjuvant was used, the amount of non-adsorbed antigen remained below 5%, even when the antigen concentration was doubled to 20 μg/ml (see Table 3). Accordingly, the use of aluminum phosphate adjuvant, with an increased HBsAg concentration, resulted in a significant and surprising increase in non-adsorbed antigen.

Accordingly, in a preferred embodiment of the disclosure, the immunogenic composition comprises between 62.5 and 500 μg/ml of aluminum as aluminum phosphate adjuvant. In a particularly preferred embodiment, the immunogenic composition of the disclosure comprises 500 μg/ml of aluminum as aluminum phosphate adjuvant and 20 μg/ml-60 μg/ml of HBsAg antigen having all three of the S, Pre-S1 and Pre-S2 proteins. In a particularly preferred embodiment, the immunogenic composition of the disclosure comprises 500 μg/ml of aluminum as aluminum phosphate adjuvant and 20 μg/ml of HBsAg antigen having all three of the S, Pre-S1 and Pre-S2 proteins.

Using in vivo studies in mice, the inventors of the present disclosure have demonstrated that the immunogenic compositions of the disclosure are effective at enhancing a Th1 response in mice. This effect is unexpected because an enhanced Th1 response was not seen in HBsAg compositions where aluminum hydroxide adjuvant was used, even when the antigen concentration was significantly increased. Specifically, as shown in Example 3, a higher 20 μg/ml concentration of HBsAg antigen (comprising S, Pre-S1 and Pre-S2 proteins) with 500 μg/ml of aluminum as aluminum hydroxide adjuvant did not enhance Th1 T cell immunity in mice compared to the commercially available prophylactic vaccine (which has 10 μg/ml of the same antigen and 500 μg/ml of aluminum as aluminum hydroxide adjuvant). However, as further described in Examples 4 and 5, a preferred immunogenic composition of the disclosure having 20 μg of antigen formulated with 500 μg/ml of aluminum from aluminum phosphate adjuvant elicited a significantly higher Th1 cell response when compared head-to-head with the prophylactic formulation of the vaccine (which has 10 μg/ml of the same antigen and 500 μg/ml of aluminum from aluminum hydroxide adjuvant). Furthermore, as described in Example 6, immunogenic compositions of the disclosure having 20 μg, 40 μg and 60 μg of antigen formulated with 500 μg/ml of aluminum from aluminum phosphate adjuvant all elicited a higher Th1 cell response when compared head-to-head with the prophylactic formulation of the vaccine (which has 10 μg/ml of the same antigen and 500 μg/ml of aluminum from aluminum hydroxide adjuvant). These results indicate that simply altering the adjuvant: antigen ratio in the preferred immunogenic composition of the disclosure was not sufficient to enhance Th1 T cell immunity. Rather, changing both the concentration of antigen and type of adjuvant (aluminum phosphate adjuvant rather than aluminum hydroxide adjuvant) was required to enhance Th1 T cell immunity. Enhanced Th1 immunity was only seen in the compositions having an increased amount of non-adsorbed antigen.

The present disclosure also provides the use of an immunogenic composition of the disclosure for the preparation of a pharmaceutical composition for inducing or enhancing a Th1 cell immune response against Hepatitis B infection in a patient. The present disclosure further provides the use of an immunogenic composition of the disclosure for the preparation of a drug for treating Hepatitis B infection, particularly chronic Hepatitis B infection, in a patient.

The present disclosure also provides pharmaceutical compositions which are useful in therapeutic applications in individuals suffering from chronic Hepatitis B infection.

In certain embodiments, provided pharmaceutical compositions may be formulated for delivery parenterally, e.g. by injection. In such embodiments, formulation may be suitable for intramuscular injection.

In some embodiments, pharmaceutical compositions are provided in a liquid dosage form that is suitable for injection. In some embodiments, pharmaceutical compositions are provided as powders (e.g. lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, gels, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). Suitable excipients include, for example, water, saline, dextrose, sucrose, trehalose, glycerol, ethanol, or similar, and combinations thereof. Remington's The Science and Practice of Pharmacy, 21st Edition, A. R. Gennaro, (Lippincott, Williams & Wilkins, Baltimore, MD, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure. In some embodiments, pharmaceutical compositions comprise one or more preservatives. In some embodiments, pharmaceutical compositions comprise no preservative.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose.

Pharmaceutical compositions described herein will generally be administered in such amounts and for such a time as is necessary or sufficient to induce or enhance a Th1 immune response in a subject. Dosing regimens may consist of a single dose or a plurality of doses over a period of time. The exact amount of a composition to be administered may vary from subject to subject and may depend on several factors. Thus, it will be appreciated that, in general, the precise dose used will be as determined by the prescribing physician and will depend not only on the weight of the subject and the route of administration, but also on the age of the subject and the severity of the symptoms. In certain embodiments, a particular amount of the vaccine composition is administered as a single dose. In certain embodiments, a particular amount of the composition is administered as more than one dose.

The present disclosure also provides a method of inducing or enhancing a T cell immune response against Hepatitis B infection in a mammal comprising administering to the mammal a composition of the disclosure. The immune response is preferably a Th1 response directed to a Hepatitis B antigen. Administration can be performed by injection by any means, for example by intramuscular injection. Injections can be made with conventional syringes and needles, or any other appropriate devices available in the art.

The present disclosure also provides a method of treating Hepatitis B infection, particularly chronic Hepatitis B infection, in a subject in need thereof comprising administering a therapeutically effective amount of a composition of the disclosure. Administration can be performed by injection by any means, for example by intramuscular injection. Injections can be made with conventional syringes and needles, or any other appropriate devices available in the art.

If desired, a method or use of the disclosure can be carried out in combination with one or more conventional therapeutic treatments for Hepatitis B infection and/or Hepatitis B-mediated disease. Administration of a composition of the disclosure may precede, be concomitant with, or subsequent to the administration of the composition of the disclosure. Therapeutic treatments which may be combined with the compositions of the disclosure may be administered for the purpose of reducing the load of Hepatitis B virus prior to or concomitant with administration of the immunogenic composition of the disclosure. Representative examples of Hepatitis B treatments which may be combined with the compositions of the disclosure include, without limitation, polymerase inhibitors, RNase H inhibitors, nucleoside analogs, nucleotide analogs, TLR agonists, N-glycosylation inhibitors, siRNA, antisense oligonucleotides, anti-hepatitis B antibodies, capsid inhibitors, core protein inhibitors, core assembly modulators, S-antigen reducers or sequesterers including nucleic acid polymers, ccc DNA inhibitors, interferons and immune modulators. Although such standard of care may vary from patient to patient, the most common Hepatitis B treatments include nucleotide or nucleoside analogs (such as lamivudine, entecavir, telbivudine, adefovir, dipivoxil or tenofovir) with or without cytokines (e.g. IFNα, pegylated IFNα2a and pegylated IFNα2b). However, newer treatments for reducing Hepatitis B viral load and/or levels of secreted HBsAg present in the plasma or serum, such as siRNA or S antigen sequestering agents (such as REP-2139, a nucleic acid polymer drug candidate produced by Replicor Inc.), may be effectively combined with the compositions of the disclosure. Hepatitis B treatments can be provided in a single dose or, alternatively, in multiple doses according to standard protocols, dosages and regimens over several hours, days weeks and/or months.

The compositions of the disclosure may also include a further adjuvant.

The invention has been described in an illustrative manner and many modifications and variations of the present invention are possible in light of the above teachings. It is therefore to be understood that within the scope of the claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### Examples

The following examples describe some exemplary modes of making and practicing certain compositions that are described herein. It should be understood that these examples are for illustrative purposes only and are not meant to limit the scope of the compositions and methods described herein.

### Example 1: Preparation of Vaccine Formulations

A Hepatitis B surface antigen consisting of all three of the S, pre-S1 and pre-S2 proteins was prepared in CHO cells in accordance with the method described in U.S. Pat. No. 5,242,812.

Vaccine formulations comprising aluminum hydroxide adjuvant were prepared as follows. Briefly, two different concentrations of HBsAg (10 mcg/ml and 20 mcg/ml) using 500 mcg/ml of aluminum content of aluminum hydroxide adjuvant (Alhydrogel®) were stirred for a time of 16±4 hours at room temperature. More specifically, phosphate sucrose buffer and HBsAg were added to a sterile glass vial and mixed gently with a pipet. To a separate sterile glass vial, a small stir bar was added followed by 0.9% saline and Alhydrogel adjuvant while stirring at 100±50 rpm. While the 0.9% saline and Alhydrogel were stirring, the mixture of PBS and HBsAg was slowly added using a 20-200 μl sized pipette (see Table 1 below). A stopper or leak-proof lid was added to each vial and securely sealed. The Alhydrogel adjuvanted vial was left stirring at room temperature (15-25° C.) for 16±4 hours at 100±50 rpm. Once stirring was completed, the vaccine formulation was stored at 2-8° C. until analysis or immunization.

TABLE 1
Immunogenic Compositions with Aluminum Hydroxide Adjuvant

| | Formulation Volumes | | | | Final Test Article | |
|---|---|---|---|---|---|---|
| | | | Volume | Volume | Target Specifications | |
| | Volume | Volume | of 0.9% | of 2% | | Final |
| | of | of | (154 mM) | Alhydrogel | | Aluminum |
| Final HBsAg | sterile | 10 μg/ml | Sodium | (10 mg/mL | Final | Concentration |
| Concentration | PBS | HBsAg | Chloride | Al+++) | Volume | (μg/mL |
| (μg/mL) | (μL) | (μL) | (μL) | (μL) | (μL) | Al+++) |
| 20 | 219 | 181 | 3400 | 200 | 4000 | 500 |
| 10 | 309 | 91 | 3400 | 200 | 4000 | 500 |

Vaccine formulations comprising aluminum phosphate adjuvant (Adjuphos®) were prepared as follows. Briefly, two different concentrations of HBsAg (10 μg/ml and 20 mcg/ml) using 500 μg/ml of aluminum as aluminum phosphate adjuvant (Adjuphos) was rotated at 8-12 rpm for 60 minutes at room temperature. More specifically, Adjuphos adjuvant was added to a sterile container followed by a 10 mM phosphate 8% sucrose buffer. HBsAg was added to the same container and mixed slowly by aspirating pipette (see Table 2 below). The container was sealed and covered with aluminum foil, and then rotated for 60 minutes at 8-12 rpm at room temperature. Once stirring was completed, the vaccine formulation were stored at 2-8° C. until analysis or immunization.

TABLE 2
Immunogenic Formulations with Aluminum Phosphate Adjuvant

| | Volume | | Volume | | |
|---|---|---|---|---|---|
| Final | of 10 mM | | of 2% | | |
| HBsAG | Phosphate | | AdjuPhos | | Final |
| Concen- | Buffer, 8% | Volume | (5 mg/mL | Final | Aluminium |
| tration | Sucrose | of HBsAg | Al+++) | Volume | Concentration |
| (μg/mL) | (μL) | (μL) | (μL) | (μL) | (μg/mL) |
| 60 | 0 | 9000* | 3000 | 30000 | 500 |
| 40 | 3000 | 6000* | 3000 | 30000 | 500 |
| 20 | 3619 | 181** | 200 | 4000 | 500 |
| 10 | 3709 | 91** | 200 | 4000 | 500 |

*HBsAg (Concentration = 200 μg/mL; Lot# B-061-6 diluted),
**HBsAg (Concentration = 441 μg/mL; Lot# B-054-3),

### Example 2: Evaluation of Adsorption of Antigen to Adjuvant

Binding of Hepatitis B surface antigen to aluminum hydroxide adjuvant and aluminum phosphate adjuvant was measured as follows. Briefly, within a sterile biosafety cabinet, 500 μL of each vaccine formulation was aseptically transferred into a sterile polypropylene centrifuge tube, after mixing 10-20 times by pipetting prior to transferring to assure the solution was homogenous. All aliquots were then centrifuged at 14,000 g for 120 minutes. Supernatant (450 μl) was carefully removed from the centrifuged tubes containing the immunogenic formulations using a 20-200 μL pipette and transferred into new, sterile polyprophylene centrifuge tubes. The centrifuged tubes containing pellets of immunogenic formulation were resuspended by addition of a volume of buffer equal to that removed (450 μl). Tubes were labelled appropriately (supernatant vs. resuspended pellets of centrifuged immunogenic formulations). Diluted pellets (containing bound HBsAg) and supernatants (containing free HBsAg) were stored at 2-8° C. in a fridge. Table 3 below demonstrates the impact of HBsAg antigen concentration, choice of aluminum adjuvant, and adjuvant dose on the amount of unbound/free HBsAg.

TABLE 3
Non-Adsorbed Antigen Content of Test HBsAg Compositions

| | Target | Target | Total Protein (μg/mL) | | | Non- |
|---|---|---|---|---|---|---|
| Test | HBsAg | Aluminum | | Supernatant | Pellet | adsorbed |
| Article | (μg/mL) | μg/mL [Al+++] | Original | (Free) | (Bound) | HBsAg |
| 1 | 40 | 62.5 Alhydrogel | 31.9 | 0.8 | 32.1 | 2.8% |
| 2 | 40 | 125 Alhydrogel | 33.2 | Not Detected | 35.8 | 0% |
| 3 | 40 | 250 Alhydrogel | 41.6 | 1.1 | 39.6 | 2.7% |
| 4 | 40 | 500 Alhydrogel | 44.8 | 1.2 | 47.6 | 2.5% |
| 5 | 20 | 500 Alhydrogel | 39.8 | Not Detected | 36.6 | 0% |
| 6 | 10 | 500 Alhydrogel | 32.5 | Not Detected | 34.3 | 0% |
| 7 | 40 | 62.5 Adjuphos | 10.2 | 5.9 | 4.9 | 54.6% |
| 8 | 40 | 125 Adjuphos | 10.1 | 5.1 | 4.2 | 54.8% |
| 9 | 40 | 250 Adjuphos | 10.5 | 4.5 | 5.2 | 46.4% |
| 10 | 40 | 500 Adjuphos | 12.2 | 3.8 | 6.8 | 35.8% |
| 11 | 20 | 500 Adjuphos | 10.0 | 3.4 | 2.8 | 54.8% |
| 12 | 10 | 500 Adjuphos | 3.5 | Not Detected | 3.1 | 0% |
| 13 | 60 | 500 Adjuphos | 11.84 | 7.6 | 8.43 | 47.4% |

Test article 6 (TA6) is the commercially available, prophylactic Sci-B-Vac vaccine and there was no detectable non-adsorbed (free) HBsAg that could be detected in the supernatant. Changing the ratio of antigen to adjuvant by increasing the antigen concentration 4-fold (TA4) did not substantially change the amount of non-adsorbed antigen (2.5%). Similarly, changing the ratio of antigen to adjuvant by reducing the amount of aluminum hydroxide adjuvant (Alhydrogel) did not increase the amount of non-adsorbed antigen above 5% (TA1-3).

Changing the adjuvant used in the immunogenic compositions from aluminum hydroxide adjuvant (Alhydrogel) (TA6) to aluminum phosphate adjuvant (Adjuphos) (TA12) similarly failed to increase the amount of non-adsorbed HBsAg (0% detected) when the concentration of HBsAg was kept at 10 μg/ml. Surprisingly, however, increasing the concentration of antigen (TA11) 2-fold and/or decreasing the concentration of aluminum phosphate adjuvant (TA7-10) substantially increased the amount of non-adsorbed HBsAg to above 30%. In particular, a 20 μg/ml concentration of antigen demonstrated a very high amount of non-absorbed HBsAg when formulated with 500 μg/ml of aluminum from aluminum phosphate adjuvant. This adjuvant concentration, specifically 500 μg/ml, is widely used in commercially available HBsAg vaccines, albeit with aluminum hydroxide adjuvant rather than aluminum phosphate adjuvant.

In summary, there is no non-adsorbed HBsAg in the commercially available prophylactic vaccine known as Sci-B-Vac (TA6) but greater than 50% of non-adsorbed HBsAg in an embodiment of the disclosure having 20 μg/ml HBsAg and 500 μg/mL aluminum as aluminum phosphate adjuvant (TA11). Changes in both the type of aluminum-based adjuvant and the concentration of antigen were necessary to obtain amounts of non-adsorbed antigen greater than 30%.

### Example 3: Evaluation of Th1 T Cell Immunity in Mice after Vaccination with Two Different Immunogenic Compositions Comprising HBsAg Comprising S, Pre-S1 and Pre-S2 and Aluminum Hydroxide Antigen

This Example describes evaluation of T cell response in mice following immunization with two different immunogenic formulations comprising different concentrations of antigen (HBsAg comprising S, Pre-S1 and Pre-S2 protein) and 500 μg/ml of aluminum from aluminum hydroxide adjuvant (TA5 and TA6 in Table 3). Balb/c mice were vaccinated 3 times on week 0, 3, 10 (or 13) with 1/20th of the human dose formulations of vaccine (i.e. 0.5 μg of antigen of TA6 and 1.0 μg of antigen of TA5). Mice were sacrificed on day 6 post 3^{rd} vaccination to measure responses to the pre-S1, pre-S2, and HBsAg S proteins using overlapping peptide pools by enzyme linked immunospot assay (“ELISPOT”) as described below. Comparable anti-HBsAg antibody responses were induced with the two formulations (data not shown).

IFN-γ ELISPOT analyses to measure Th1 T cell responses were performed as follows. Mice were split into groups of 8 mice each immunized with the two test formulations described above, each having the same concentration of aluminum hydroxide adjuvant but different concentrations of HBsAg antigen. Four mice per group were sacrificed and spleens were removed. Spleens from individual mice were processed to produce single cell suspensions. Erythrocytes were lysed using a commercially available buffer (BioLegend). Splenocytes were then re-suspended at 6×10⁶ splenocytes/mL. One day prior to spleen collection and processing, ELISPOT plates were coated with Interferon-gamma (IFN-γ) capture antibody. No peptide, actin peptide mix, Pre-S1 peptide mix, Pre-S2 peptide mix, HBsAg peptide mix, and phorbol 12-myristate 13-acetate and ionomycin (PMA/iono) were selected as stimulants. On the day of the spleen collection, stimulants were added to designated ELISPOT plate wells. 1.5×10⁵ splenocytes were added to PMA/iono wells and 3×10⁵ splenocytes were added to all other stimulants. The ELISPOT plates were then placed into a humid 37° C. with 5% CO2 incubator for 40-48 hours. After incubation, the plates were washed for removal of splenocytes, stimulants and media and IFN-γ capture antibody was added, followed by streptomycin horseradish peroxidase (strep-HRP). The plates were finally developed with commercially available 3-Amino-9-ethylcarbazole (AEC) substrate (BD BioSciences). The observed spots were counted using an ELISPOT plate reader and the final data was reported as spot forming cells (SFC) per one million splenocytes. Table 4 below presents the results of this immunogenicity study.

TABLE 4
Th1 Activity as measured by ELISPOT

| | Pre-S1 | | Pre-S2 | | sAg | |
|---|---|---|---|---|---|---|
| | (IFN-γ + SFC/10⁶ | | (IFN-γ + SFC/10⁶ | | (IFN-γ + SFC/10⁶ | |
| Immunogenic | splenocytes) | | splenocytes) | | splenocytes) | |
| Composition | GM | SE | GM | SE | GM | SE |
| 10 μg HBsAg | 6.048 | 2.549 | 124.6 | 38.31 | 1056 | 225.9 |
| Formulation | | | | | | |
| containing 500 | | | | | | |
| μg/mL [Al+++] | | | | | | |
| (Alhydrogel) | | | | | | |
| 20 μg HBsAg | 6.328 | 2.918 | 112.3 | 48.17 | 1094 | 214.6 |
| Formulation | | | | | | |
| containing 500 | | | | | | |
| μg/mL[Al+++] | | | | | | |
| (Alhydrogel) | | | | | | |

Note:
GM = Geometric mean;
SE = Standard Error

Table 4 demonstrates that higher amounts of HBsAg antigen in formulations containing aluminum hydroxide as the adjuvant did not show improved Th1 T cell response. This result is consistent with the data in Table 2, which showed that both formulations had no non-absorbed antigen.

### Example 4: Comparison of T Cell Immunity in Mice Following Vaccination with a Commercially Available Prophylactic Vaccine Known as Sci-B-Vac (10 μg/Ml HBsAg Comprising S, Pre-S1 and Pre-S2 and 500 μg/Ml Aluminum as Aluminum Hydroxide Adjuvant) and a Formulation of the Disclosure Comprising 20 μg HBsAg and 500 μg/Ml Aluminum as Aluminum Phosphate Adjuvant

This Example describes evaluation of T cell response in mice following vaccination with two different immunogenic compositions. The first composition was the commercially available prophylactic vaccine known as Sci-B-Vac which comprises 10 μg/ml HBsAg comprising S, Pre-S1 and Pre-S2 proteins and 500 μg/mL aluminum as aluminum phosphate adjuvant (TA6 in Table 3). The second composition was an immunogenic composition of the disclosure comprising a 2-fold higher concentration of the same HBsAg (20 μg/ml) and 500 μg/mL aluminum as aluminum phosphate adjuvant (TA11 in Table 3). Balb/c mice were vaccinated 3 times on week 0, 3, 6 (or 8) with 1/20th of the human dose of each immunogenic composition (i.e. 0.5 μg of antigen of TA6 and 1.0 μg of antigen of TA11). Mice were sacrificed on day 6 post 3^{rd} vaccination to measure responses to the pre-S1, pre-S2, and HBsAg proteins using overlapping peptide pools by ELISPOT as described above. Comparable anti-HBsAg antibody responses were induced with the two formulations.

TABLE 5
Th1 Activity as measured by ELISPOT

| | Pre-S1 | | Pre-S2 | | sAg | |
|---|---|---|---|---|---|---|
| | (IFN-γ + SFC/10⁶ | | (IFN-γ + SFC/10⁶ | | (IFN-γ + SFC/10⁶ | |
| Immunogenic | splenocytes) | | splenocytes) | | splenocytes) | |
| Composition | GM | SE | GM | SE | GM | SE |
| 10 μg HBsAg | 5.833 | 0.4951 | 53.66 | 7.991 | 160.3 | 35.78 |
| Formulation | | | | | | |
| containing 500 | | | | | | |
| μg/mL[Al+++] | | | | | | |
| (Alhydrogel) | | | | | | |
| 20 μg HBsAg | 5.439 | 0.8325 | 271.7 | 62.9 | 333.0 | 35.51 |
| Formulation | | | | | | |
| containing 500 | | | | | | |
| μg/mL[Al+++] | | | | | | |
| (Adjuphos) | | | | | | |

As can be seen from Table 5, the immunogenic composition of the disclosure containing 20 μg HBsAg and 500 μg/ml aluminum as aluminum phosphate adjuvant stimulates a substantially greater Th1 cell response against the Pre-S2 and S antigens than does the commercially available prophylactic version of the vaccine formulated with a much lower concentration of the same antigen (10 μg/ml) and aluminum hydroxide adjuvant. This result is consistent with the data in Table 2 which shows a large percentage of non-bound antigen (54.8%) in the immunogenic composition of the disclosure (TA11) as compared to no unbound antigen in the commercially available prophylactic HBsAg vaccine (TA6). The response appears to have been greatest with respect to Pre-S2. No responses were detected against the preS1 antigen with either formulation.

### Example 5: Comparison of Individual IgG1/IgG2a Ratios in Mice Following Vaccination with a Commercially Available Prophylactic Vaccine Known as Sci-B-Vac (10 μg/Ml HBsAg Comprising S, Pre-S1 and Pre-S2 and 500 μg/Ml Aluminum as Aluminum Hydroxide Adjuvant) and a Formulation of the Disclosure Comprising 20 μg HBsAg and 500 μg/Ml Aluminum as Aluminum Phosphate Adjuvant

This Example describes comparison of individual IgG1/IgG2a ratios in mice following vaccination with two different immunogenic compositions. The first composition was the commercially available prophylactic vaccine known as Sci-B-Vac which comprises 10 μg/ml HBsAg comprising S, Pre-S1 and Pre-S2 proteins and 500 μg/mL aluminum as aluminum hydroxide adjuvant (TA6 in Table 3). The second composition was an immunogenic composition of the disclosure comprising a 2-fold higher concentration of the same HBsAg (20 μg/ml) and 500 μg/mL aluminum as aluminum phosphate adjuvant (TA11 in Table 3). Balb/c mice (n=8) were vaccinated 3 times on weeks 0, 3, 6 with 1/20th of the human dose of each were sacrificed on day 6 post 3^{rd} vaccination to measure Anti-HBs IgG1 and Anti-HB IgG2 by ELISA as follows.

Anti-HBs IgG1:96 well plates were coated overnight at 4° C., with recombinant Hepatitis B Surface antigen Protein, Abcam (0.25 μg/mL in DPBS). On the following day, plates were blocked with 10% goat sera in ELISA wash buffer (0.05% Tween-20 in PBS) for 1 hour at 37° C. Plates were washed with wash buffer, followed by addition of 2-fold dilutions of individual mouse sera; starting at 1:10,000 to 1:1280,000. Plates were incubated, for 1.5 hours at 37° C., followed by plate washing and addition of secondary antibody. Goat anti-mouse IgG1 (Bethyl), diluted 1:10,000 in 10% goat sera in ELISA wash buffer was added and the plates were incubated for 1.5 hours at 37° C. TMB One component Microwell substrate was added to the plates, incubated at room temperature for 10 minutes and then Stop solution was added. Absorbance was read at 450 nm using a MAXline plate reader.

Anti-HBs IgG2a: 96 well plates were coated overnight at 4° C., with Recombinant Hepatitis B Surface antigen Protein, Abcam (0.25 μg/ml in DPBS). On the following day, plates were blocked with 10% goat sera in ELISA wash buffer, for 1 hour at 37° C. Plates were washed with wash buffer, followed by addition of 2-fold dilutions, of individual mouse sera; starting at 1:5,000 to 1:640,000. Plates were incubated, for 1.5 hours at 37° C., followed by plate washing and addition of secondary antibody. Goat anti-mouse IgG2a (Bethyl), diluted 1:10,000 in 10% goat sera in ELISA wash buffer was added and the plates were incubated for 1.5 hours at 37° C. TMB One component Microwell substrate was added to the plates and incubated at room temperature for 10 minutes and then Stop solution was added. Absorbance was read at 450 nm using a MAXline plate reader.

The results are shown in Table 6 below.

TABLE 6
Individual Ig G1/IgG2 Ratios

| | Ratio IgG1/IgG2a | Ratio IgG1/IgG2a |
|---|---|---|
| | 10 μg HBsAg | 20 μg HBsAg |
| Individual | Formulation containing | Formulation containing |
| Mouse | 500 μg/mL[Al+++] | 500 μg/mL [Al+++] |
| number | (Alhydrogel) | (Adjuphos) |
| 1 | 5.3 | 2.5 |
| 2 | 36.5 | 3.6 |
| 3 | 118.3 | 5.6 |
| 4 | 19.9 | 0.1 |
| 5 | 35.9 | 6.9 |
| 6 | 2.0 | 4.6 |
| 7 | 20.5 | 0.9 |
| 8 | N/A | 9.4 |

As can be seen from Table 6, the immunogenic composition of the disclosure containing 20 μg HBsAg and 500 μg/ml aluminum as aluminum phosphate adjuvant stimulates a significantly lower ratio of IgG1 to IgG2a. Since IgG2a stimulation is a marker for Th1 activity, this altered ratio indicates that the composition containing 20 μg HBsAg and 500 μg/ml aluminum as aluminum phosphate adjuvant elicits a stronger Th1 response than the composition comprising 20 μg HBsAg and 500 μg/ml aluminum as aluminum phosphate adjuvant.

### Example 6: Dose Ranging Study in Mice Following Vaccination with a Commercially Available Prophylactic Vaccine Known as Sci-B-Vac (10 μg/Ml HBsAg Comprising S, Pre-S1 and Pre-S2 and 500 μg/Ml Aluminum as Aluminum Hydroxide Adjuvant) and Three Different Doses of the Composition of the Disclosure Comprising 20 μg, 40 μg and 60 μg HBsAg and 500 μg/Ml Aluminum as Aluminum Phosphate Adjuvant

This Example describes a dose ranging study which examines the Th1 activity in mice following vaccination with the commercially available prophylactic vaccine known as Sci-B-Vac which comprises 10 μg/ml HBsAg comprising S, Pre-S1 and Pre-S2 proteins and 500 μg/mL aluminum as aluminum hydroxide adjuvant (TA6 in Table 3) and three different doses comprising of the same HBsAg immunogenic composition of the disclosure and 500 μg/mL aluminum as aluminum phosphate adjuvant. The three different doses were as follows HBsAg (20 μg/ml) (TA11 in Table 3); HBsAg (40 μg/ml) (TA10 in Table 3) and HBsAg (60 μg/ml) (TA 13 in Table 3).

Briefly, Balb/c mice (n=8/group) were vaccinated intramuscularly 3 times, 3 weeks apart with a mouse dose of each the compositions, specifically 3 μg, 2 μg and 1 μg. Splenocytes were harvested from mice 7 days after the 3rd vaccination and stimulated with overlapping peptide pools specific to preS1, Pre-S2, and HBsAg. The IFN-γ-secreting T cell responses were evaluated by ELISPOT after 48 hours of culture as follows. One day prior to spleen collection and processing, ELISpot plates (Millipore) were coated with 100 μl Interferon-gamma (IFN-γ) capture antibody at a concentration of 15 μg/mL (Mabtech) and incubated overnight at 4° C.

On the day of spleen collection and processing, the coated ELISpot plates were washed 5 times with 200 μl sterile PBS and blocked with 100 μl of R10 media for 1-2 hrs. Once the splenocytes had been isolated and counted, the R10 blocking media was removed and 50 μl of splenocytes (300,000 cells) and 50 μl of the stimulants were plated onto the ELISpot assay plates. Splenocytes from each mouse were stimulated in duplicate with following stimulants: PreS1 (final stimulation concentration=13.5 μg/ml), Pre-S2 (final stimulation concentration=5.5 μg/ml) and HBsAg (final stimulation concentration=27 μg/mL), R10 as a negative control and phorbol 12-myristate 13-acetate and ionomycin (PMA (20 ng/ml)/Ionomycin (1 μg/ml) as a positive control. The ELISpot plates were then placed into a humid 37° C. with 5% CO2 incubator for 40-48 hours. After incubation, the plates were washed 5 times with 200 μl PBS-Tween for removal of splenocytes, stimulants and media and 100 μl of IFN-γ capture antibody (Mabtech) at a concentration of 1 μg/ml was then added to each well. Following a 2 hour incubation, the ELISpot plates were washed 5 times with PBC-Tween and 100 μl streptomycin horseradish peroxidase (strep-HRP) diluted 1:1000 was added to each well. The plates were then incubated for a further hour before being developed for 30 minutes at room temperature by adding 100 μL 3-Amino-9-ethylcarbazole (AEC) substrate (BD BioSciences). The observed spots were counted by ZellNet Consulting and the final data reported as spot forming units (SFC) per one million splenocytes. The Th1 activity of the different compositions as measured by ELISPOT are shown in Table 7.

TABLE 7
Th1 Activity as Measured by ELISPOT

| | Pre-S1 | | Pre-S2 | | sAg | |
|---|---|---|---|---|---|---|
| | (IFN-γ + SFC/10⁶ | | (IFN-γ + SFC/10⁶ | | (IFN-γ + SFC/10⁶ | |
| Immunogenic | splenocytes) | | splenocytes) | | splenocytes) | |
| Composition | M | SE | M | SE | M | SE |
| 20 μg HBsAg | 1.665 | 1.044 | 261.2 | 62.43 | 498.0 | 125.4 |
| Formulation | | | | | | |
| containing 500 | | | | | | |
| μg/mL[Al+++] | | | | | | |
| (Adjuphos) | | | | | | |
| 40 μg HBsAg | 0.9514 | 0.4951 | 222.6 | 69.20 | 649.6 | 109.1 |
| Formulation | | | | | | |
| containing 500 | | | | | | |
| μg/mL[Al+++] | | | | | | |
| (Adjuphos) | | | | | | |
| 60 μg HBsAg | 0.7136 | 0.7136 | 148.9 | 31.01 | 422.0 | 102.0 |
| Formulation | | | | | | |
| containing 500 | | | | | | |
| μg/mL[Al+++] | | | | | | |
| (Adjuphos) | | | | | | |
| 10 μg HBsAg | 0.0 | 0.0 | 108.4 | 36.95 | 226.4 | 28.54 |
| Formulation | | | | | | |
| containing 500 | | | | | | |
| μg/mL[Al+++] | | | | | | |
| (Alhydrogel) | | | | | | |

As can be seen in Table 7, the 20 μg and 40 μg and 60 μg dose of the compositions comprising HBsAg comprising S, Pre-S1 and Pre-S2 proteins and 500 μg/mL aluminum as aluminum phosphate adjuvant were able to elicit greater numbers of Pre-S2 and HBsAg specific IFN-γ-secreting T cells by ELISPOT when compared to the 10 μg/ml HBsAg comprising S, Pre-S1 and Pre-S2 proteins and 500 μg/mL aluminum as aluminum hydroxide adjuvant (TA6 in Table 3). Accordingly, each of the three doses of the immunogenic compositions of the disclosure induced a higher T cell response that the composition comprising 10 μg/ml HBsAg comprising S, Pre-S1 and Pre-S2 proteins and 500 μg/mL aluminum as aluminum hydroxide adjuvant.

### OTHER EMBODIMENTS

Other embodiments of the disclosure will be apparent to those skilled in the art from a consideration of the specification or practice of the disclosure disclosed herein. It is intended that the specification and examples be considered as exemplary only, with the true scope of the disclosure being indicated by the following claims. The contents of any reference that is referred to herein are hereby incorporated by reference in their entirety.

1-15. (canceled)

16. An immunogenic composition comprising:
(a) an HBsAg antigen comprising S protein, Pre-S1 protein and Pre-S2 protein; and
(b) an aluminum phosphate adjuvant,
wherein the composition comprises at least 20 μg/ml of HBsAg antigen and the weight ratio of the HBsAg antigen to the amount of aluminum present in the adjuvant is from about 40:62.5 to about 20:500.

17. The immunogenic composition according to claim 16, wherein the weight ratio is about 20:500.

18. The immunogenic composition according to claim 16, wherein the weight ratio is about 40:500.

19. The immunogenic composition according to claim 16, wherein the weight ratio is 60:500.

20. The immunogenic composition according to claim 16, wherein the aluminum is present in a concentration of about 500 μg/ml.

21. The immunogenic composition according to claim 16, wherein the HBsAg antigen in a concentration of about 20 μg/ml.

22. The immunogenic composition according to claim 16, wherein the HBsAg antigen in a concentration of about 40 μg/ml.

23. The immunogenic composition according to claim 16, wherein the HBsAg antigen in a concentration of about 60 μg/ml.

24. A pharmaceutical composition comprising the immunogenic composition of claim 16 and a pharmaceutically acceptable excipient.

25. A method of inducing a Th1 cell response in a mammal, said method comprising administering the immunogenic composition of claim 16.

26. A method of treating Hepatitis B in a subject, said method comprising administering a therapeutically effective amount of the immunogenic composition of claim 16 to the subject.

27. The method of claim 26, wherein an additional Hepatitis B treatment is administered to the subject prior to, concurrently with, or after administration of the immunogenic composition to the subject.

28. The method of claim 27, wherein the additional Hepatitis B treatment is a nucleoside inhibitor or a pegylated interferon alpha.

29. The method of claim 27, wherein the additional Hepatitis B treatment is a polymerase inhibitor, an RNase H inhibitor, a TLR agonist, an N-glycosylation inhibitor, an antisense oligonucleotide, an anti-hepatitis B antibody, a capsid inhibitor, a core protein inhibitor, a core assembly modulator, an S-antigen reducer or sequesterer, a nucleic acid polymers, a ccc DNA inhibitor, an interferon, an immune modulator or an siRNA.

30. An aqueous pharmaceutical composition comprising
(a) an HBsAg antigen in a concentration of at least 20 μg/ml HBsAg antigen; and
(b) an aluminum phosphate adjuvant,
wherein the aluminum is present in a concentration of at least 500 μg/ml and the amount of non-adsorbed antigen is at least 30% in the aqueous pharmaceutical composition.

31. The aqueous pharmaceutical composition according to claim 30, wherein the HBsAg antigen comprises an S protein having at least 90% identity to the amino acid sequence of SEQ ID NO:1.

32. The aqueous pharmaceutical composition according to claim 30, wherein the HBsAg antigen comprises S protein, Pre-S1 protein, and Pre-S2 protein.

33. The aqueous pharmaceutical composition according to claim 32, wherein the HBsAg antigen comprises 75-90% by weight S protein, 2-8% by weight Pre-S1 protein, and 5-15% by weight Pre-S2 protein.

34. The aqueous pharmaceutical composition according to claim 30, wherein the ratio of the HBsAg antigen to the aluminum present in the adjuvant is 60:500.

35. The aqueous pharmaceutical composition according to claim 30, wherein the ratio of the HBsAg antigen to the aluminum present in the adjuvant is 40:500.

36. The aqueous pharmaceutical composition according to claim 30, wherein the ratio of the HBsAg antigen to the aluminum present in the adjuvant is 20:500.

37. A method of inducing a Th1 cell response in a mammal, said method comprising administering aqueous pharmaceutical composition of claim 30.

38. A method of treating Hepatitis B in a subject, said method comprising administering a therapeutically effective amount of the aqueous pharmaceutical composition of claim 30 to the subject.

39. The method of claim 38, wherein an additional Hepatitis B treatment is administered to the subject prior to, concurrently with, or after administration of the aqueous pharmaceutical composition to the subject.

40. The method of claim 39, wherein the additional Hepatitis B treatment is a nucleoside inhibitor or a pegylated interferon alpha.

41. The method of claim 39, wherein the additional Hepatitis B treatment is a polymerase inhibitor, an RNase H inhibitor, a TLR agonist, an N-glycosylation inhibitor, an antisense oligonucleotide, an anti-hepatitis B antibody, a capsid inhibitor, a core protein inhibitor, a core assembly modulator, an S-antigen reducer or sequesterer, a nucleic acid polymers, a ccc DNA inhibitor, an interferon, an immune modulator or an siRNA.


SEQUENCE LISTING (3 sequences)

SEQ ID NO: 1 - protein, 226 aa, synthetic construct
  source 1..226: note = Description of Artificial Sequence: Synthetic polypeptide
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWGSVRFSWLSLLVPFVQWFVGLSPTVWLSVIWMMWYWGPNLYNILSPFIPLLPIFFCLWVYI

SEQ ID NO: 2 - protein, 281 aa, synthetic construct
  source 1..281: note = Description of Artificial Sequence: Synthetic polypeptide
MQWNSTAFHQALQHPRVRGLYFPAGGSSSGTVNPAQNIASHISSISSRTGDPAPNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWGSVRFSWLSLLVPFVQWFVGLSPTVWLSVIWMMWYWGPNLYNILSPFIPLLPIFFCLWVYI

SEQ ID NO: 3 - protein, 399 aa, synthetic construct
  source 1..399: note = Description of Artificial Sequence: Synthetic polypeptide
MGLSWTVPLEWGKNQSTSNPLGFFPDHQLDPAFGANSNNPDWDLNSNKDHWPQANQVGVGAFGPGFTPPHGGLLGWSSQAQGTLHTVPAVPPPASTNRQTKRQPTPISPPLRDSHPQAMQWNSTAFHQALQHPRVRGLYFPAGGSSSGTVNPAQNIASHISSISSRTGDPAPNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWGSVRFSWLSLLVPFVQWFVGLSPTVWLSVIWMMWYWGPNLYNILSPFIPLLPIFFCLWVYI